(19) United States
(12) Patent Application Publication
(10) Pub. No.: US 2024/0050291 A1
(43) Pub. Date: Feb. 15, 2024

(54) Fibrous Web and Surge Layer Made from Same

(71) Applicant: Kimberly-Clark Worldwide, Inc., Neenah, WI (US)

(72) Inventors: Roger Quincy, Roswell, GA (US); Steven C. Lavy, Loganville, GA (US); Carl Wust, Conyers, GA (US)

(21) Appl. No.: 18/257,017

(22) PCT Filed: Dec. 13, 2021
(86) PCT No.: PCT/US2021/063058

§ 371 Date: Jun. 12, 2023

Related U.S. Application Data
(60) Provisional application No. 63/132,762, filed on Dec. 31, 2020.

(51) Int. Cl. A61F 13/537; D04H 1/541; D04H 3/009; D04H 1/4391; D04H 1/4291; D04H 3/018; D04H 1/542

(52) U.S. Cl. CPC A61F 13/537; D04H 1/5412; D04H 3/009; D04H 1/43914; D04H 1/4291; D04H 3/018; D04H 1/542; D10B 2509/026; A61F 2013/530226

(57) ABSTRACT

Nonwoven webs having excellent fluid handling characteristics are disclosed. The nonwoven webs are made from a combination of binder fibers and structure fibers. The nonwoven webs can be made exclusively from polyolefin polymers without having to contain polyester fibers. Although the webs can be used in numerous applications, the nonwoven materials are particularly well suited as a surge layer in an absorbent article.

### BACKGROUND

Desired performance objectives for personal care absorbent products include low leakage from the product and a dry feel to the wearer. However, absorbent articles commonly fail before the total absorbent capacity of the product has been utilized. Absorbent garments, such as incontinence garments and disposable diapers, often leak at the legs and waist. The leakage can be due to a variety of shortcomings in the product, one being insufficient fluid uptake by the absorbent system, especially on the second or third liquid insults.

For example, the initial uptake rates for conventional absorbent structures can deteriorate once they have already received liquid surges into their structures. The disparity between liquid delivery and uptake rates can result in excessive pooling on the surface of the fabric before the liquid is taken-up by the absorbent core. During this time, pooled liquid can leak from the leg openings of the diaper and soil the outer clothing and bedding of the wearer. Attempts to alleviate leakage have included providing physical barriers with such design features as elastic leg gathers and changing the amount and/or configuration of the absorbent material in the zone of the structure into which the liquid surges typically occur.

Nonwoven materials such as carded webs and spunbonded webs have been used as the body side liners in absorbent products. Specifically, very open, porous liner structures have been employed to allow liquid to rapidly pass through them and to help keep the body skin separated from the wetted absorbent pad underneath the liner.

In addition to using a porous body side liner, many absorbent articles are further equipped with a surge layer. Surge layers can be made with thick, lofty fabric structures that possess a significant amount of void space. The surge layers are positioned between the body side liner and the absorbent structure. Surge layers are designed to rapidly absorb liquids in order to move the liquids away from the body so that they can be absorbed by the absorbent structure. Surge layers can provide the personal care absorbent products with faster fluid intake and better dryness.

In the past, surge layers were typically made from polyester fibers. The polyester fibers gave the layer resilience and compression resistance that allowed the materials to retain significant amounts of void space. The use of polyester fabrics within the absorbent article, however, presents problems. For example, the polyester materials create significant recycling issues, particularly since the remainder of the absorbent article is made from other polymers, such as polyolefins.

In this regard, a need currently exists for an improved surge material that can be made from polyolefin polymers. A need also exists for a surge material that does not contain any polyester fibers.

### SUMMARY

In general, the present disclosure is directed to a surge material made from polyolefin fibers that has the characteristics of conventional materials made from polyester fibers. Nonwoven webs made according to the present disclosure, for instance, have high loft characteristics, possess a significant amount of void space, and are well suited to rapidly absorbing liquids for moving the liquids from the body side liner to the absorbent structure.

For instance, in one embodiment, the present disclosure is directed to an absorbent article comprising a body side liner, an outer cover, and an absorbent structure positioned between the body side liner and the outer cover. In accordance with the present disclosure, the absorbent article further includes a surge layer positioned between the body side liner and the absorbent structure. The surge layer comprises a nonwoven web containing a blend of binder fibers and structure fibers. The structure fibers are designed to provide loft and void volume. The binder fibers are designed to hold the structure together and provide strength. The structure fibers comprise polypropylene staple fibers having a linear density (dtex) of from about 8 dtex to about 14 dtex. The binder fibers form bond sites within the surge layer at crossover locations where the binder fibers intersect with other fibers and at other locations.

In one aspect, the polypropylene staple fibers have a linear density of from about 8.5 dtex to about 10.5 dtex. The polypropylene staple fibers can also comprise hollow fibers. The structure fibers can be present in the nonwoven web in an amount from about 20% by weight to about 60% by weight, such as in an amount from about 35% by weight to about 55% by weight. The binder fibers, on the other hand, can be present in the nonwoven web in an amount from about 40% by weight to about 80% by weight, such as in an amount from about 45% by weight to about 65% by weight. In one aspect, a greater amount of binder fibers is present in the nonwoven web in relation to the structure fibers on a weight percentage basis. In addition, the nonwoven web can be constructed without containing any polyester fibers.

In one aspect, the binder fibers comprise bicomponent fibers. The binder fibers can be made from one or more polyolefin polymers. For example, in one embodiment, the binder fibers include a core made from a polypropylene polymer surrounded by a sheath made from a polyethylene polymer. In one aspect, the binder fibers have a linear density or size of from about 3 dtex to about 6 dtex. In another aspect, the binder fibers can have a linear density of from about 6.5 dtex to about 12.5 dtex. The binder fibers can have an elongation of greater than about 170%, such as greater than about 175%.

The nonwoven web that forms the surge layer in the absorbent article can generally have a basis weight of from about 30 gsm to about 90 gsm, such as from about 40 gsm to about 60 gsm. In one aspect, the nonwoven web can be a carded web. In addition, the nonwoven web can be through-air bonded for causing the binder fibers to bond to intersecting fibers without compressing the web.

The binder fibers and the structure fibers can have any suitable length that provides void volume and integrity. In one aspect, the binder fibers and the structure fibers can have an average length of from about 38 mm to about 65 mm, such as from about 45 mm to about 60 mm. In order to improve the fluid handling properties of the surge material, in one embodiment, the fibers in the nonwoven web can be treated with a hydrophilic finish.

In addition to absorbent articles, the present disclosure is also directed to nonwoven webs having fluid management properties. The nonwoven web can be made from a blend of binder fibers and structure fibers as described above. The nonwoven web can be used not only in absorbent articles, but in other applications where fluid handling characteristics are desired. For instance, the nonwoven web can also be used as a filter element in filter devices.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a perspective view of one embodiment of a nonwoven web made in accordance with the present disclosure;

FIG. 2 is a perspective view with cutaway portions of one embodiment of a feminine care product made in accordance with the present disclosure;

FIG. 3 is a perspective view of another embodiment of an absorbent article made in accordance with the present disclosure;

FIG. 4 is a graphical representation of some of the results obtained in the example described below; and

FIG. 5 is another graphical representation of some of the results obtained in the example below.

Repeat use of reference characters in the present specification and drawings is intended to represent the same or analogous features or elements of the present invention.

### Definitions

As used herein the term “nonwoven fabric or web” refers to a web having a structure of individual polymeric and/or cellulosic fibers or threads that are interlaid, but not in an identifiable manner as in a knitted fabric. Nonwoven fabrics or webs have been formed from many processes such as for example, meltblowing processes, spunbonding processes, bonded carded web processes, those used to make tissue and towels, etc.

As used herein the term “staple fiber” means fibers that have a fiber length generally in the range of about 5 millimeters to about 150 millimeters. Staple fibers can be cellulosic fibers or non-cellulosic fibers. Some examples of suitable non-cellulosic fibers that can be used include, but are not limited to, hydrophilically-treated polyolefin fibers, polyester fibers, nylon fibers, polyvinyl acetate fibers, and mixtures thereof. Hydrophilic treatments can include durable surface treatments and treatments in polymer resins/blends. Cellulosic staple fibers include for example, pulp, thermomechanical pulp, synthetic cellulosic fibers, modified cellulosic fibers, and the like. Cellulosic fibers can be obtained from secondary or recycled sources. Synthetic cellulosic fibers such as, for example, rayon, viscose rayon, and lyocell can be used. Modified cellulosic fibers are generally composed of derivatives of cellulose formed by substitution of appropriate radicals (e.g., carboxyl, alkyl, acetate, nitrate, etc.) for hydroxyl groups along the carbon chain.

As used herein “bonded carded webs” refer to nonwoven webs formed by carding processes as are known to those skilled in the art and further described, for example, in U.S. Pat. No. 4,488,928 to Ali Khan et al., which is incorporated herein by reference thereto. Briefly, carding processes involve starting with a blend of, for example, staple fibers with binder fibers or other bonding components in a bulky ball that is combed or otherwise treated to provide a generally uniform basis weight. This web is heated or otherwise treated to activate the adhesive component resulting in an integrated, usually lofty nonwoven material.

As used herein, the term “hydrophilic” generally refers to fibers or films, or the surfaces of fibers or films that are wettable by aqueous liquids in contact with the fibers. The term “hydrophobic” includes those materials that are not hydrophilic as defined. The phrase “naturally hydrophobic” refers to those materials that are hydrophobic in their chemical composition state without additives or treatments affecting the hydrophobicity.

The degree of wetting of the materials can, in turn, be described in terms of the contact angles and the surface tensions of the liquids and materials involved. Equipment and techniques suitable for measuring the wettability of particular fiber materials or blends of fiber materials can be provided by the Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

As used herein, the terms “personal care product” and “absorbent article” refer to any article capable of absorbing water or other fluids. Examples of some absorbent articles include, but are not limited to, personal care absorbent article such as diapers, training pants, absorbent underpants, adult incontinence products including fitted briefs, belted shields, guards for men, protective underwear, adjustable underwear, feminine hygiene products (e.g., sanitary napkins, pad, liners, and the like), swim wear, and so forth. Materials and processes suitable for forming such absorbent articles are well known to those skilled in the art.

Disposable absorbent products are designed to be removed and discarded after a single use. By single use it is meant that the disposable absorbent incontinence product will be disposed of after being used once instead of being laundered or cleaned for reuse, as is typical of regular cloth underwear.

As used herein, the linear density of a fiber is measured in dtex, which is the grams of fiber per ten kilometers and is a direct measure of linear density. Linear density can also be measured in denier, which is the mass of grams of the fiber per 9,000 meters.

As used herein, the “draw” or the “draw ratio” of a fiber is defined as the ratio of the final length of the fiber to the original length or unoriented length of the fiber after stretching the fiber.

The elongation of a fiber and the tenacity of a fiber, which is a measure of the fiber's specific strength, are measured according to ASTM Test D76 and/or ASTM Test D2101.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a fibrous material that has excellent fluid handling characteristics. The fiber material has significant strength and integrity and yet possesses significant void volume for absorbing fluids. The fiber material can be used in all different types of applications. In one particular embodiment, the fiber material can be used as a surge layer in an absorbent article.

Resilient surge layers provide personal care absorbent articles with faster fluid intake and better dryness. Effective surge layers, for instance, are capable of rapidly absorbing liquids and transferring the liquids to an absorbent core that is designed to store the liquids away from the skin of the user. Surge layers work in conjunction with the absorbent core in keeping the wearer dry and preventing leaks from occurring through the waist and legs. For example, surge layers have fluid handling properties that can effectively disperse liquids that hit and saturate a particular target insult area. Surge layers increase the capability of the absorbent article to move liquid away from the target insult area in order to limit saturation and improve the overall fluid handling performance of the article, especially during multiple insults.

In general, absorbent articles include a body side liner, an outer cover, and an absorbent core positioned between the body side liner and the outer cover. Surge layers are positioned between the absorbent core and the body side liner in order to provide fluid handling benefits. In the past, effective surge materials typically required great amounts of polyester fibers. Incorporating polyester fibers into an absorbent article, however, can significantly impact the ability to recycle the absorbent article, in that the article is made primarily from polyolefin polymers. In this regard, the present disclosure is generally directed to a nonwoven material that can be used as a surge layer in an absorbent article, that is made primarily from polyolefin polymers and has excellent fluid handling properties.

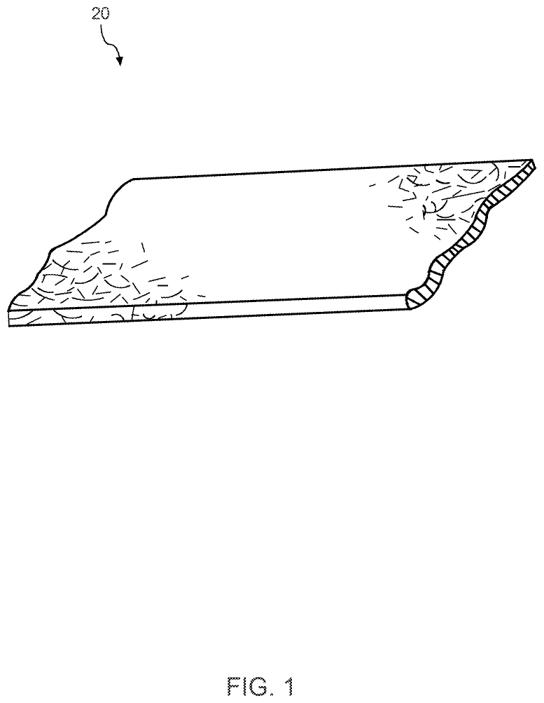

As described above, the material of the present disclosure that can be used as a surge layer is a fiber material in the form of a nonwoven web. Referring to FIG. 1, for instance, for exemplary purposes only, a nonwoven web 20 made in accordance with the present disclosure is shown. The nonwoven web 20 contains at least two different fiber types. More particularly, the nonwoven web 20 includes structure fibers combined with binder fibers. In one aspect, the nonwoven web 20 is made exclusively from the structure fibers and binder fibers and contains no other fiber or filler material. The structure fibers and the binder fibers can both be made from polyolefin polymers.

In one aspect, the nonwoven web 20 can be a carded web, particularly a bonded carded web. In this regard, the nonwoven web 20 can be made from structure fibers and binder fibers that both are staple fibers. In producing a carded web, bales of the fibers can optionally be placed in contact with a picker which separates the fibers. Next, the fibers are fed through a combing or carding unit which further breaks apart and optionally aligns the staple fibers along a direction, such as the machine direction (lengthwise direction) so as to form a fibrous nonwoven web. Once the web is formed, the web is then bonded using one or more bonding methods. In accordance with the present disclosure, the web contains binder fibers which facilitate bonding of the fibers where they intersect in order to give the web integrity and strength. In one aspect, for instance, the carded web can be bonded using through-air bonding. Through-air bonding, for instance, controls the level of compression or collapse of the nonwoven web during the bonding process. In through-air bonding, heated air is forced through the web to melt at least one component within the web to cause bonding sites to form. The component that is melted can be a portion of the binder fibers. For example, the binder fibers can include a polyethylene polymer and a polypropylene polymer where the lower melting polyethylene component forms a sheath that surrounds a polypropylene core. The sheath polymer can melt during through-air bonding and cause the binder fibers to bond to other fibers at crossover locations in the web where the binder fibers intersect other fibers. During through-air bonding, the nonwoven web can be supported on a forming wire or drum. In addition, optionally a vacuum may be pulled through the web in order to better control the process.

Through the process as described above, the structure fibers within the web become bonded to the binder fibers and provide a voided structure within the web that greatly enhances liquid absorption.

The structure fibers selected for use in the fiber material of the present disclosure are polyolefin fibers having a selected size and tenacity that has been found to greatly increase the fluid handling properties of the web. More particularly, the structure fibers are designed to have a combination of fiber size, fiber tenacity and elongation and be bonded in the web in a way that creates a web having polyester-like performance.

For instance, in one aspect, the structure fibers can comprise polypropylene staple fibers. In one aspect, for instance, the structure fibers are hollow polypropylene staple fibers. The polypropylene polymer used to form the fibers can be a polypropylene homopolymer or a polypropylene copolymer. Polypropylene copolymers that may be used include random copolymers of polypropylene and an alpha-olefin monomer, such as ethylene, butylene, or the like. The structure fibers can generally have an average fiber length of from about 38 mm to about 65 mm, including all increments of one mm therebetween. For instance, the structure fibers can have an average fiber length of greater than about 45 mm, such as greater than about 50 mm, such as greater than about 52 mm, and generally less than about 60 mm.

The structure fibers, in one aspect, can have a relatively high linear density or fiber size. For instance, the structure fibers can have a linear density of from about 8 dtex to about 14 dtex, including all increments of 0.1 dtex therebetween. For example, the linear density of the structure fibers can be greater than about 8 dtex, such as greater than about 8.2 dtex, such as greater than about 8.4 dtex, such as greater than about 8.6 dtex, such as greater than about 8.8 dtex, such as greater than about 9 dtex, such as greater than about 9.2 dtex, such as greater than about 9.4 dtex. The linear density of the structure fibers is generally less than about 14 dtex, such as less than about 13 dtex, such as less than about 12 dtex, such as less than about 11 dtex, such as less than about 10 dtex, such as less than about 9.8 dtex.

In one aspect, the structure fibers are drawn in order to increase tenacity and/or adjust the elongation of the fibers. For example, the structure fibers can have a draw ratio of greater than about 3, such as greater than about 3.5, such as greater than about 3.75, and generally less than about 6, such as less than about 5. In one aspect, the structure fibers are drawn but are not crimped.

The structure fibers can generally have a tenacity of greater than about 4.25 cN, such as greater than about 4.5 cN, such as greater than about 4.75 cN, such as greater than about 5 cN. The tenacity of the fibers is generally less than about 8 cN, such as less than about 6 cN. Fibers having the above tenacity characteristics generally have an elongation of less than about 115%, such as less than about 105%, such as less than about 100%, such as less than about 95%, such as less than about 90%. The elongation of the structure fibers is generally greater than about 45%, such as greater than about 55%, such as greater than about 65%, such as greater than about 75%, such as greater than about 80%.

The structure fibers can be present in the nonwoven web 20 as shown in FIG. 1 generally in an amount of from about 20% by weight to about 60% by weight, including all increments of 1% therebetween. For instance, the structure fibers can be present in the nonwoven web 20 in an amount greater than about 30% by weight, such as in an amount greater than about 35% by weight, such as in an amount greater than about 40% by weight, such as in an amount greater than about 45% by weight, and generally in an amount less than about 60% by weight, such as less than about 55% by weight.

As described above, the structure fibers are combined with binder fibers to construct the nonwoven web 20. The binder fibers can also be constructed from polyolefin fibers. In general, the binder fibers contain a polyolefin polymer at the surface of the fiber that has a melting temperature that is lower than the melting temperature of the polymer used to produce the structure fibers.

Similar to the high loft staple fibers, the binder fibers can also be staple fibers having an average fiber length of from about 38 mm to about 65 mm. For instance, the binder fibers can have an average fiber length of greater than about 40 mm, such as greater than about 45 mm, and generally less than about 60 mm, such as less than about 55 mm.

In one aspect, the binder fibers can be monocomponent fibers made from a single polymer. The polymer used to produce the binder fibers, for instance, can be a polyethylene polymer.

In an alternative embodiment, the binder fibers are bicomponent fibers. The bicomponent fibers, for instance, can have a high melting point component or polymer combined with a lower melting point component or polymer in a side-by-side arrangement or in a sheath/core arrangement. In a sheath and core arrangement, for instance, the higher melting point component forms the core of the fiber while the lower melting point polymer or component forms the sheath of the fiber. The lower melting point component provides an efficient means for bonding the fibers to other fibers while the higher melting point component aids in maintaining the structural integrity of the fiber.

In one aspect, the higher melting point component or core can be made from a polypropylene polymer. The polypropylene polymer can be a polypropylene homopolymer or a random copolymer containing polypropylene. The random copolymer can be, for instance, a copolymer of propylene and butylene or a copolymer of propylene and ethylene.

The sheath or surface polymer, on the other hand, can comprise a polyethylene polymer, such as a linear low density polyethylene or high density polyethylene polymer. In still another embodiment, the sheath polymer can be a random copolymer of ethylene and propylene.

In a sheath and core arrangement, the core polymer generally comprises from about 20% to about 80% by weight of the fiber, such as in an amount from about 40% to about 60% by weight. Similarly, the sheath polymer can be present in the fiber in an amount from about 20% to about 80% by weight, such as in an amount from about 40% to about 60% by weight.

The binder fibers incorporated into the nonwoven web 20 as shown in FIG. 1 can also be drawn fibers. For instance, the binder fibers can have a draw ratio of greater than about 2, such as greater than about 2.4, and generally less than about 5, such as less than about 4, such as less than about 3.5. The linear density of the fibers can vary depending upon the particular application. In general, the linear density can be from about 3 dtex to about 12.5 dtex, including all increments of 0.5 dtex therebetween. In one aspect, higher linear density fibers may be used in which the binder fibers have a linear density of from about 6.5 dtex to about 12.5 dtex. Alternatively, smaller sized binder fibers may be used having a linear density of from about 3 dtex to about 6 dtex.

The binder fibers generally have a tenacity of greater than about 2 cN, such as greater than about 2.25 cN, and generally less than about 4.5 cN, such as less than about 4 cN, such as less than about 3.5 cN. The binder fibers generally have an elongation of less than about 350%, such as less than about 325%, such as less than about 300%, such as less than about 290%, and generally greater than about 150%, such as greater than about 170%, such as greater than about 175%, such as greater than about 180%.

The binder fibers are generally present in the nonwoven web 20 as shown in FIG. 1 in an amount from about 40% by weight to about 80% by weight, including all increments of 1% by weight therebetween. For example, the binder fibers can be present in the nonwoven web 20 in an amount greater than about 45% by weight, such as in an amount greater than about 50% by weight, such as in an amount greater than about 55% by weight, and generally less than about 70% by weight, such as in an amount less than about 65% by weight.

The structure fibers and the binder fibers can be blended together to form the nonwoven web 20 as shown in FIG. 1. In one aspect, the fibers can be well blended such that the nonwoven web 20 has a substantially homogeneous fiber distribution. The basis weight of the resulting nonwoven web 20 can vary depending upon the particular application. Nonwoven webs made according to the present disclosure can be used in all different types of applications including use as a surge layer in an absorbent article, can be used as a filter layer in a filter device, or can be used in various other applications. In general, the basis weight can be from about 12 gsm to about 250 gsm, including increments of 1 gsm therebetween. When used as a surge layer, for instance, the nonwoven web 20 can have a basis weight of generally greater than about 30 gsm, such as greater than about 35 gsm, such as greater than about 40 gsm, such as greater than about 45 gsm, such as greater than about 50 gsm, and generally less than about 110 gsm, such as less than about 90 gsm, such as less than about 80 gsm, such as less than about 70 gsm, such as less than about 60 gsm.

In one aspect, the nonwoven web as shown in FIG. 1 can further include a hydrophilic treatment in order to further improve the fluid handling properties of the web. The hydrophilic treatment agent of the present disclosure can be selected from the group consisting of polyethylene glycol laurates, polyethylene glycol lauryl ethers, and combinations thereof. Examples of suitable polyethylene glycol laurates include, but are not limited to, polyethylene glycol 400 monolaurate, polyethylene glycol 600 monolaurate, polyethylene glycol 1000 monolaurate, polyethylene glycol 4000 monolaurate, polyethylene glycol 600 dilaurate, and combinations thereof. Examples of suitable polyethylene glycol lauryl ethers include, but are not limited to, polyethylene glycol 600 lauryl ether.

In addition to the PEG laurates and PEG lauryl ethers, other polyethylene glycol derivatives can be used as hydrophilic treatment agents for the personal care products described herein. As used herein, the term “polyethylene glycol derivative” includes any compound including a polyethylene glycol moiety. Examples of other suitable PEG derivatives include, but are not limited to, PEG monostearates such as PEG 200 monostearates and PEG 4000 monostearate; PEG dioleates such as PEG 600 dioleate and PEG 1540 dioleate; PEG monooleates such as PEG 600 monooleate and PEG 1540 monooleate; PEG monoisostearates such as PEG 200 monoisostearate; and PEG 16 octyl phenyl.

In certain aspects, the hydrophilic treatment agents described herein, such as polyethylene glycol 600 lauryl ether and/or the polyethylene glycol 600 monolaurate, can be used in combination with each other or in combination with other viscoelastant agents. Examples of additional viscoelastant agents that can be used in combination with the hydrophilic treatment agents include, but are not limited to, sodium citrate, dextran, cysteine, Glucopon 220UP (available as a 60% (by weight) solution of alkyl polyglycoside in water from Henkel Corporation), Glucopon 425, Glucopon 600, Glucopon 625. Other suitable viscoelastant agents are described in U.S. Pat. No. 6,060,636.

The hydrophilic treatment agent can be applied in varying amounts depending on the desired results and application. Typically, the hydrophilic treatment agent is applied to the web in an amount of from about 0.1% to about 40%, from about 0.1% to about 20%, or from about 3% to about 12%, or alternatively from about 0.3% to about 1.5% by weight of the treated substrate. The hydrophilic treatment can be applied to the fibers or to the nonwoven material. In one aspect, the hydrophilic treatment can comprise an aqueous solution containing a hydrophilic agent that is applied to the fibrous material using a kiss roll or other suitable method. For instance, the hydrophilic treatment can also be sprayed on the fibrous material.

As described above, the nonwoven web 20 as shown in FIG. 1 is particularly well suited for use as a surge layer in an absorbent article. The nonwoven web can be used in all different types of personal care absorbent articles including, but not limited to, diapers, training pants, incontinence garments, sanitary napkins, bandages, and the like.

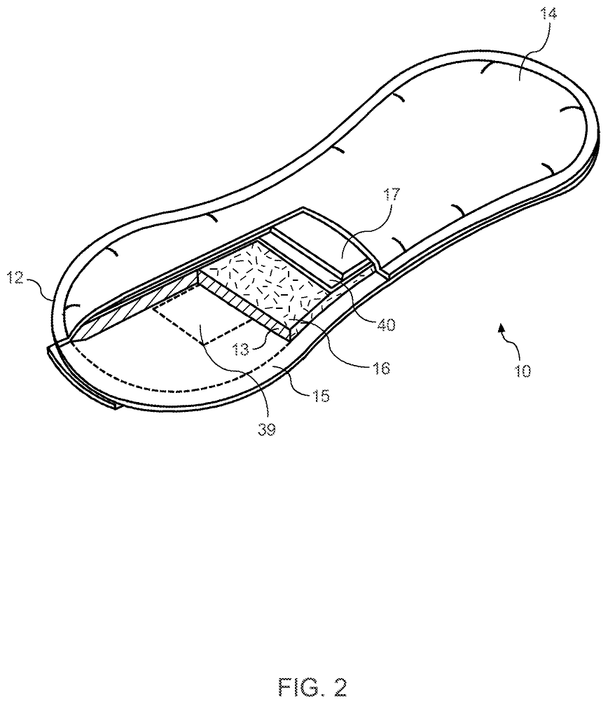

For example, disposable absorbent articles include feminine hygiene pads such as the pad 10 shown in FIG. 2. Pad 10 includes a bodyside liner 14 and a baffle or outer cover 15 that extend to a pad perimeter 12. The pad 10 can include an absorbent core 13 and a transfer or surge layer 17 made in accordance with the present disclosure disposed between the bodyside liner 14 and the baffle or outer cover 15. The absorbent core 13 can include an optional core wrap 16. In an aspect of the present disclosure, the pad 10 can include a dispersion layer 40 positioned between the transfer or surge layer 17 and the absorbent core 13. Many products also have an adhesive strip 39 to help hold the product in place during use by adhering it to the user's underclothes.

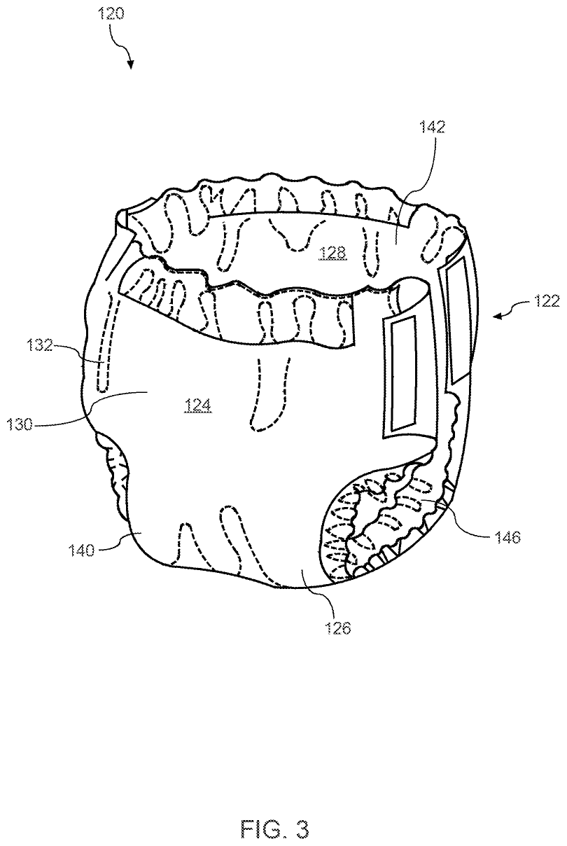

The disposable absorbent article can also be a diaper or training pant, such as the training pant shown in FIG. 3 in a partially fastened condition. The pant 120 defines a pair of longitudinal end regions, otherwise referred to herein as a front region 122 and a back region 124, and a center region, otherwise referred to herein as a crotch region 126, extending longitudinally between and interconnecting the front and back regions 122, 124. The pant 120 also defines an inner surface 128 adapted in use (e.g., positioned relative to the other components of the pants 120) to be disposed toward the wearer, and an outer surface 130 opposite the inner surface. The illustrated pant 120 includes a chassis 132 that includes an outer cover 140 and a bodyside liner 142 that can be joined to the outer cover 140 in a superimposed relation therewith by adhesives, ultrasonic bonds, thermal bonds or other conventional techniques. The chassis 132 can further include a surge layer in accordance with the present disclosure (not shown) and an absorbent structure (not shown) disposed between the outer cover 140 and the bodyside liner 142 for absorbing liquid body exudates exuded by the wearer, and can further include a pair of containment flaps 146 secured to the bodyside liner 142 for inhibiting the lateral flow of body exudates.

The surge layer in accordance with the present disclosure can help absorb, decelerate, and diffuse surges or gushes of liquid that may be rapidly introduced into the absorbent article as shown in either FIG. 2 or FIG. 3. The surge layer is generally located between the body side liner and the absorbent core. In one aspect, the surge layer can be attached to one or more of the various components in the absorbent article such as the absorbent core, the body side liner, or a wrap that may be surrounding the absorbent core.

The outer cover of the absorbent article can be made from a liquid impermeable material. For example, in one aspect, the outer cover can be formed from a spunbond nonwoven web such as a spunbond polypropylene nonwoven web, from a film such as a polyolefin film, or from a laminate of the above materials.

The body side liner, on the other hand, is liquid permeable and can be made from materials that are suitably compliant and soft feeling when placed adjacent to the wearer's skin. The body side liner can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner. For example, the body side liner can be made from a meltblown or spunbonded web of polyolefin fibers. The body side liner can also be a bonded-carded web composed of natural and/or synthetic fibers.

A suitable liquid permeable body side liner is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent structure, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an air laying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material.

The present disclosure may be better understood with reference to the following example.

### Example

Various different bonded carded webs were constructed and tested as a surge layer in an absorbent article.

In constructing the webs, the following bicomponent fibers and structure fibers were used:

### Bicomponent Binder Fibers

| | | Linear | | |
|---|---|---|---|---|
| | | density | | |
| Fiber No. | Draw Ratio | (dtex) | Tenacity (cN) | Elongation (%) |
| A1 | 2.6X | 6.0 | 1.78 | 355 |
| A2 | 2.6X | 6.0 | 2.35 | 283 |
| A3 | 3.3X | 4.8 | 3.07 | 184 |
| A4 | 3.3X | 6.1 | 2.85 | 168 |
| A5 | 3.3x | 10.7 | 2.42 | 279 |

### Structure Fibers

| | | Linear | | |
|---|---|---|---|---|
| | | density | | |
| Fiber No. | Draw Ratio | (dtex) | Tenacity (cN) | Elongation (%) |
| B1 | 3.25X | 7.2 | 4.10 | 120 |
| B2 | 3.25X | 7.4 | 4.50 | 108 |
| B3 | 4.00X | 5.9 | 5.83 | 45 |
| B4 | 4.00X | 7.7 | 5.25 | 87 |
| B5 | 4.00X | 9.1 | 5.13 | 86 |

The bicomponent binder fibers above included a polypropylene core surrounded by a polyethylene sheath. The structure fibers were comprised of polypropylene hollow fibers.

Through-air bonded, carded webs were constructed using the fibers identified above. In particular, 60% by weight of a binder fiber was combined with 40% by weight of a high loft fiber to produce a bonded carded web having a basis weight of 50 gsm. More particularly, the following nonwoven webs were created:

| Sample No. | Fiber Content | | | |
|---|---|---|---|---|
| 3 | 60% | A1 | 40% | B1 |
| 4 | 60% | A2 | 40% | B2 |
| 5 | 60% | A2 | 40% | B5 |
| 6 | 60% | A4 | 40% | B5 |
| 7 | 60% | A5 | 40% | B5 |
| 8 | 60% | A3 | 40% | B5 |
| 9 | 60% | A3 | 40% | B3 |
| 10 | 60% | A4 | 40% | B4 |
| 11 | 60% | A2 | 40% | B3 |

The above nonwoven webs were then incorporated into an absorbent article and tested for fluid intake and rewet. The webs were compared to two different nonwoven webs made with 40% polyester (PET) fiber in place of the polypropylene hollow fiber having the same basis weight. Sample No. 1, for instance, was a nonwoven polyester web containing polyester fibers having a linear density of 10.2 dtex. Sample No. 2 was a nonwoven web made from polyester fibers having a linear density of 16.7 dtex.

All of the nonwoven samples were made with fibers treated with a hydrophilic finish in an amount of from about 0.35% by weight to about 0.6% by weight. Each of the samples was placed in an absorbent article between a body side liner and an absorbent core. The body side liner was a hydrophilic treated 12 gsm spunbond-meltblown-spunbond web.

The following test was conducted to determine fluid intake and rewet.

### Abstract

This test categorizes the amount of fluid remaining near the surface of the diaper shortly after insult, as well as quantifies the amount of fluid not locked up by superabsorbent under high pressure after a longer wait and multiple insults. For successful usage, the product must both intake fluid quickly through the layers of the absorbent core, in addition to holding on to fluid to ensure that it does not flow back out when subjected to high pressure. The volume of loadings and the rate of fluid delivery are predefined based on previous consumer studies with the product. These values can vary from product to product.
1.0 Equipment and Supplies
1.1 Top loading electronic balance capable of reading 0.001 gram.
1.2 Saline solution, 0.9±0.005% (w/w) aqueous isotonic saline.
1.3 Countdown timer, readable to 0.1 second
1.4 Rectangular Plexiglass plate (Dimensions length=300 mm and width=100 mm) including an open cylinder located in the central area of the plate (the internal diameter of the cylinder is 38 mm and the height is 125 mm)
1.5 Two weights of 4 kg each
1.6 Blotter Paper Verigood grade, white, 100 Ib, 475 by 600 mm (19 by 24 inches) long stock, 250 sheets per ream, cut to a specified size of 88×300 mm+/−13 mm (3.5 by 12 inches)
1.7 Polycarbonate plate (3 675 mm thick) cut to 114 mm wide×432 mm long (4 5 by 17 inches) and weighing 177 grams.
1.8 Funnel Polyethylene, 4 ounce capacity
1.9 Low tack two-sided tape or attachment material to secure product flat on surface.
1.10 Stopwatch, readable to 0.1 second
1.11 Ruler
2.0 Sample Preparation
2.1 Based on the product size determine insult size and rate from table below
2.2 Weigh product to the nearest 0.01 grams and record, discard specimens out of weight range determined by requestor if applicable
3.0 Setup Procedure
3.1 Ensure saline is room temperature
3.2 Have three countdown timers set for 30 seconds, 2 minutes, and 15 minutes
4.0 Test Procedure
4.1 Place the product on a flat plane and fix the top and bottom ends to the two-sided tape so that the product is stretched and the absorbent core lays flat.
4.2 Place the board with open cylinder on the stretched product so that the top edge of the plate is aligned with the edge of the absorbent core. Put the funnel in the top of the open cylinder in the plate.
4.3 Weigh one sheet of blotter paper on the balance readable to three decimal places. Record the weight of the paper.
4.4 Pour the specified quantity of saline solution into the funnel in accordance with the size of the product being tested by following the instructions in the table below. Simultaneously activate the stopwatch.
4.5 Stop the stopwatch as soon as the liquid passes completely from the cylinder and into the product (no liquid being on the surface of the product). Start the timer set to 30 seconds and the timer set to 15 minutes.
4.6 Record the intake time.
4.7 After waiting 30 seconds, remove the plate with the open cylinder. Place the pre-weighed blotter paper on the product and place the polycarbonate board on top of it.
4.8 Start the timer set to 2 minutes.
4.9 After waiting 2 minutes, remove the polycarbonate board and blotter paper. Place the plate with the open cylinder on the product again and leave on the product for the duration of the wait.
4.10 Weigh the blotter paper to the nearest thousandth, and record the weight.
4.11 Mark the two ends down the length of the blotter paper that the fluid extends to. Measure that distance in centimeters lengthwise down the blotter paper and widthwise. Multiply those two distances together to get the area (spread) of the fluid. Record spread.
4.12 After the 15 minute wait, place the one weight on both sides of the cylinder on the board. Pour the specified quantity of saline solution, depending on the table below, into the funnel placed in the cylinder. Simultaneously start the stopwatch, and start the countdown timer set to 15 minutes.
4.13 Stop the stopwatch as soon as the liquid passes completely through the cylinder and into the product (no liquid being on the surface of the product). Record the second intake time. Leave the plate with the open cylinder and weights on the product for the duration of wait time.
4.14 Weigh two sheets of blotter paper, and record the weight to three decimal places.
4.15 After the 15 minute wait, remove the weights and rectangular board with cylinder. Place the pre-weighed blotter paper onto the product, and then place the polycarbonate board and the two weights on top of that. Start the timer set for 2 minutes.
4.16 After the 2 minute wait, remove the weights, polycarbonate board, and blotter paper. Immediately weigh the blotter paper on a scale readable to three decimal places and record the weight.
4.17 Mark the two ends down the length of the blotter paper that the fluid extends to. Measure that distance in centimeters lengthwise down the blotter paper and widthwise. Multiply those two distances together to get the area (spread) of the fluid. Record spread.
4.18 Remove the product from the surface. Weigh the product and record the weight.

| | | | | | | | | | |
|---|---|---|---|---|---|---|---|---|---|
| Product Size | N | Step 1 | Step 2 | Step 3 | Step 4 | Step 5 | 2T/3T | 3T/4T | 4T/5T |
| Rate (ml/s) | 9 ml/s | 10 ml/s | 12 ml/s | 15 ml/s | 15 ml/s | 15 ml/s | 15 ml/s | 15 ml/s | 15 ml/s |
| Volume (ml) | 40 | 50 | 60 | 70 | 85 | 100 | 80 | 100 | 110 |
| Target | 4 | 5.5 | 6.5 | 7.5 | 8.5 | 9.5 | 8.5 | 9 | 9.5 |
| location (cm) | | | | | | | | | |
| (measured | | | | | | | | | |
| from center) | | | | | | | | | |

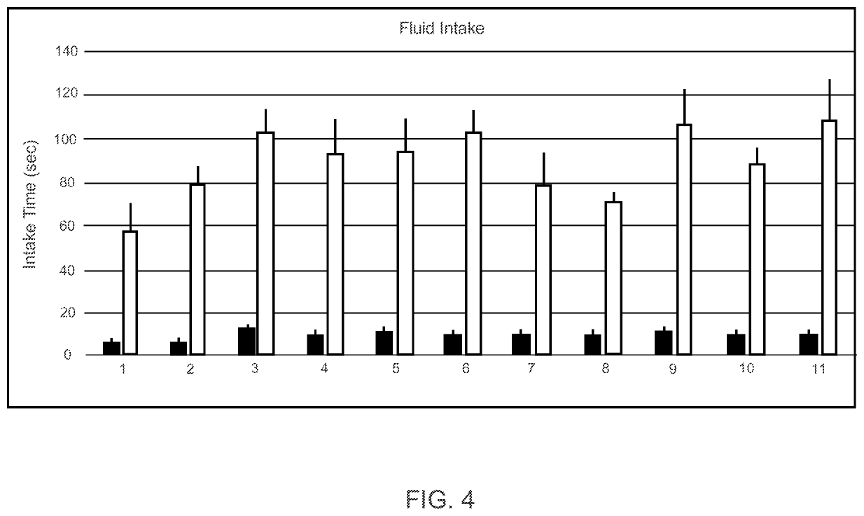

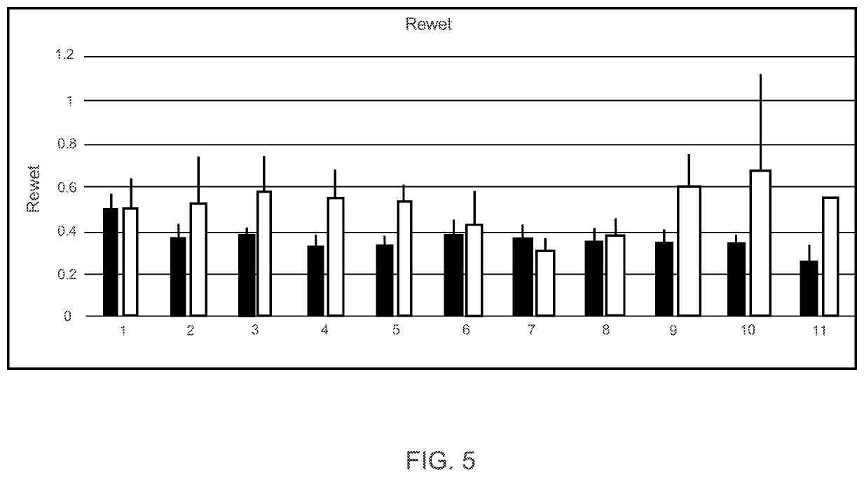

FIGS. 4 and 5 illustrate the results obtained from the fluid intake and rewet test. As shown in FIGS. 4 and 5, Sample Nos. 7 and 8 demonstrated superior fluid handling characteristics and compare favorably to Sample Nos. 1 and 2 that contain polyester fibers.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An absorbent article comprising:
a body side liner;
an outer cover;
an absorbent structure positioned between the body side liner and the outer cover; and
a surge layer positioned between the body side liner and the absorbent structure, the surge layer comprising a nonwoven web comprising a blend of binder fibers and structure fibers, the structure fibers comprising polypropylene staple fibers having a linear density of from about 8 dtex to about 14 dtex, the binder fibers forming bond sites within the surge layer.

2. An absorbent article as defined in claim 1, wherein the polypropylene staple fibers have a linear density of from about 8.5 dtex to about 10.5 dtex.

3. An absorbent article as defined in claim 1, wherein the polypropylene staple fibers comprise hollow fibers.

4. An absorbent article as defined in claim 1, wherein the structure fibers are present in the nonwoven web in an amount from about 20% to about 60% by weight, the binder fibers being present in the nonwoven web in an amount from about 40% to about 80% by weight.

5. An absorbent article as defined in claim 1, wherein the nonwoven web does not contain any polyester fibers.

6. An absorbent article as defined in claim 1, wherein the binder fibers comprise bicomponent fibers.

7. An absorbent article as defined in claim 6, wherein the bicomponent fibers are made from polyolefin polymers.

8. An absorbent article as defined in claim 6, wherein the bicomponent fibers include a sheath layer surrounding a core, the sheath layer comprising a polyethylene polymer, the core comprising a polypropylene polymer.

9. An absorbent article as defined in claim 6, wherein the binder fibers have a linear density of from about 3 dtex to about 6 dtex.

10. An absorbent article as defined in claim 6, wherein the binder fibers have a linear density of from about 6.5 dtex to about 12.5 dtex.

11. An absorbent article as defined in claim 6, wherein the binder fibers have a tenacity of greater than about 2 cN and less than about 4.5 cN.

12. An absorbent article as defined in claim 6, wherein the binder fibers have an elongation of greater than about 170%.

13. An absorbent article as defined in claim 1, wherein the nonwoven web has a basis weight of from about 30 gsm to about 90 gsm.

14. An absorbent article as defined in claim 1, wherein the nonwoven web comprises a carded and through-air bonded web.

15. An absorbent article as defined in claim 1, wherein the binder fibers and the structure fibers have an average fiber length of from about 38 mm to about 65 mm.

16. An absorbent article as defined in claim 1, wherein the surge layer further comprises a hydrophilic finish applied to fibers contained in the nonwoven web.

17. A nonwoven material having fluid management properties comprising:
a nonwoven web comprising a blend of binder fibers and structure fibers, the structure fibers comprising polypropylene hollow staple fibers having a linear density of from about 8 dtex to about 14 dtex, the binder fibers comprising staple bicomponent fibers, the binder fibers forming bond sites within the nonwoven web at crossover locations where the binder fibers intersect other fibers, the nonwoven web comprising a carded web having a basis weight of from about 30 gsm to about 90 gsm, the nonwoven web containing no polyester fibers.

18. A nonwoven material as defined in claim 17, wherein the polypropylene staple fibers have a linear density of from about 8.5 dtex to about 10.5 dtex.

19. A nonwoven material as defined in claim 17, wherein the bicomponent fibers include a sheath layer surrounding a core, the sheath layer comprising a polyethylene polymer, the core comprising a polypropylene polymer.

20. A nonwoven material as defined in claim 17, wherein the binder fibers and the structure fibers have an average fiber length of from about 38 mm to about 65 mm.

21. A nonwoven material as defined in claim 17, wherein the surge layer further comprises a hydrophilic finish applied to fibers contained in the nonwoven web.
